(19) United States
(12) Patent Application Publication
(10) Pub. No.: US 2022/0137019 A1
(43) Pub. Date: May 5, 2022

(54) HYPERSPECTRAL COMPUTER VISION AIDED TIME SERIES FORECASTING FOR EVERY DAY BEST FLAVOR

(71) Applicant: Walmart Apollo, LLC, Bentonville, AR (US)

(72) Inventors: Tarun Dasari, Bentonville, AR (US); Aditya Reddy Cheruku, Bentonville, AR (US); Anand Banik, Rogers, AR (US); Sumanth B. Talasila, Bentonville, AR (US); Kishore P. Sasthiri, Bentonville, AR (US); Murthy N. Kolluru, Bentonville, AR (US)

(21) Appl. No.: 17/626,782

(22) PCT Filed: Jul. 14, 2020
(86) PCT No.: PCT/US2020/041896

§ 371 Date: Jan. 12, 2022

Related U.S. Application Data
(60) Provisional application No. 62/874,264, filed on Jul. 15, 2019.

(51) Int. Cl. G01N 33/02; G06Q 10/06; G01N 21/27; G01N 21/25

(52) U.S. Cl. CPC G01N 33/025; G06Q 10/06395; G01N 2021/8466; G01N 21/255; G01N 21/27

(57) ABSTRACT

In some embodiments, apparatuses and methods are provided herein useful to determining a flavor profile for an item. In some embodiments, a computing system for determining a flavor profile of an item comprises a memory device storing computer-executable instructions and a processor configured to execute the computer-executable instructions to obtain a spectral profile associated with the item, identify at least one attribute value for at least one attribute of the item based on the received spectral profile, determine a flavor score for the item based on the at least one attribute value, obtain time series data associated with the item corresponding to a number of days, calculate a predicted flavor score for the item relative to the number of days based on the received time series data, and generate a flavor profile for the item based at least on the predicted flavor score.

### CROSS-REFERENCE TO RELATED APPLICATION

This application is national stage entry of International Application PCT/US2020/041896, which claims the benefit of U.S. Provisional Application No. 62/874,264, filed Jul. 15, 2019, each of which is incorporated by reference in its entirety herein.

### TECHNICAL FIELD

This invention relates generally to retail products and, more particularly product quality assessment.

### BACKGROUND

Flavor of an item is one factor in building consumer loyalty and managing inventory. Flavor may vary between items of the same type based on a number of factors. With produce, for example, certain attributes, such as crunchiness, acidity, and sugar levels, may aid in determining the flavor of the produce, and its appeal to consumers. Other characteristics may be less obvious but affect how an item is perceived and its desirability. Flavor, however, is not static, but rather changes over time and may be affected by other factors such as temperature, humidity, pressure, and other aspects of the environment. The change in flavor affects whether a consumer will desire the item, and also affects how the item is sold.

### SUMMARY

Examples of the disclosure provide for a computing system for generating a flavor profile for an item, the computing system comprising a memory device storing computer-executable instructions configured to obtain sensor data, including a spectral profile associated with the item; determine at least one attribute value for at least one attribute of the item based on the received sensor spectral profile data; determine a flavor score for the item based on the at least one attribute value; obtain time series data associated with the item corresponding to a number of days; calculate a predicted flavor score for the item relative to the number of days based on the received time series data; and generate a flavor profile for the item based at least on the predicted flavor score.

Still other examples provide one or more computer storage devices storing computer-executable instructions for obtaining a spectral profile associated with the item; determining at least one attribute and a corresponding score of the at least one attribute of the item based on the obtained spectral profile; obtaining time series data associated with the item corresponding to a number of days; calculating a predicted attribute score of the at least one attribute based on the corresponding score of the attribute and the received time series data; determining a flavor score of the item based on the calculated predicted attribute score; and generating a flavor profile for the item based at least on the flavor score.

This summary is provided to introduce a selection of concepts in a simplified form that are further described below in the Detailed Description. This summary is not intended to identify key features or essential features of the claimed subject matter, nor is it intended to be used as an aid in determining the scope of the claimed subject matter.

### BRIEF DESCRIPTION OF THE DRAWINGS

Disclosed herein are embodiments of systems, apparatuses, and methods pertaining to systems, methods, and apparatuses for determining a flavor profile of an item. This description includes drawings, wherein:

FIG. 1 is a simplified diagram of embodiments of the systems and methods described herein in accordance with the disclosure;

FIG. 2 is an exemplary computing device configured to perform embodiments of the systems and methods described herein in accordance with the disclosure;

FIG. 3 is an exemplary block diagram of a process for determining flavor of an item in accordance with the disclosure;

FIG. 4 is an exemplary flow chart of a process for determining flavor of an item in accordance with the disclosure;

FIG. 5 is an exemplary illustration of a device identifying flavor scores and related information about an item in accordance with the disclosure;

FIG. 6 is an exemplary chart of other embodiments of an output of a flavor predictor in accordance with the disclosure; and

FIG. 7 is an exemplary embodiment of a computing environment for implementing processes and methods described herein in accordance with the disclosure.

Elements in the figures are illustrated for simplicity and clarity and have not necessarily been drawn to scale. For example, the dimensions and/or relative positioning of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention. Certain actions and/or steps may be described or depicted in a particular order of occurrence while those skilled in the art will understand that such specificity with respect to sequence is not actually required. The terms and expressions used herein have the ordinary technical meaning as is accorded to such terms and expressions by persons skilled in the technical field as set forth above except where different specific meanings have otherwise been set forth herein.

### DETAILED DESCRIPTION

Generally speaking, pursuant to various embodiments, systems, apparatuses, and methods are provided herein useful to determining a flavor profile of an item. In some embodiments, a computing system for determining a flavor profile of an item comprises a memory device storing computer-executable instructions and a processor configured to execute the computer-executable instructions to obtain a spectral profile associated with the item, identify at least one attribute value for at least one attribute of the item based on the received spectral profile, determine a flavor score for the item based on the at least one attribute value, obtain time series data associated with the item corresponding to a number of days, calculate a predicted flavor score for the item relative to the number of days based on the received time series data, and generate a flavor profile for the item based at least on the predicted flavor score.

Predicting flavor of an item may be helpful in selling an item and optimizing inventory management of the item. Providing flavor predictions of an item over the duration of the shelf life of the item may increase consumer satisfaction with the item. In order to predict flavor, particularly over a period of time, various attributes of an item may be measured, and information related to how an item is affected by time and environmental conditions may be obtained and analyzed to predict a flavor score for the item. This prediction of flavor forms the basis of a flavor profile that may be provided to consumers.

In some embodiments, each attribute (e.g., crunchiness, acidity, sugar level, and the like) of a particular item, may be identified using a spectral profile. The spectral profile can be formed based on an image, such as a spectral image, of the item. In the example of a spectral profile, the image itself does not show the attribute. Rather, the image shows various shades, colors, heat signatures, and the like at different parts of the item. These characteristics may then be processed to determine various attributes of the item. Each attribute may then receive a specific score associated with the initial scan or image capture. A combination of these attribute scores may be referred to as a spectral profile. In some examples, the obtained spectral profile is analyzed together with time series data to predict how the attribute will change over a period of time.

Time series data may comprise data showing the various characteristics or attribute changes over time for an item or item type, such as the rate at which an item loses its firmness over several days for example. Time series data may be combined with analysis methods in order to extract statistics and other characteristics of the data. Time series data may be item-specific historical or machine learned data regarding how an item changes over time and in response to a variety of environmental factors. In addition to time series data, other data such as environmental data associated with the item may be measured and used with the time series data to generate a prediction of a forecasted flavor for the item.

In one example, when a customer at a store obtains an image of an item, such as a fruit or vegetable, using a mobile device, a flavor profiler may use the image to determine attributes of the item. The flavor profiler may be implemented on the mobile device capturing the image or may be communicatively coupled to the mobile device and implemented remote from the mobile device, such as in a cloud computing system. The flavor profiler may score the various attributes of the item and may use the attribute scores to create a flavor score for the item. The flavor score may then be used with stored or retrieved data related to the item or similar items, such as time series data, environmental data, and the like, to forecast a flavor for the item over its shelf life and generate a flavor profile for the item. The flavor profile may be output to a display device or other data access mechanism, such as a barcode. In some examples, the flavor profile may be dynamic and may be updated periodically as different scans or data associated with the item is obtained. In some cases, a flavor profile prediction may be generated for each of a number of days, such as for each day the item is in transport.

In other embodiments, the attributes themselves may be forecasted over time. In such instances, the flavor score may be determined at any point in time from the current or forecasted attributes. The determined flavor score and the scores of the attributes may together form the flavor profile. Additionally, the flavor profile may comprise other information corresponding to the item, such a point of origin, time in transit, and the like. The flavor profile may be customizable by a user and configured to provide any desired sub-set of available information the user deems appropriate.

The flavor profiler and time series data may also be refined over time by repeatedly scanning an item at different stages and points in time. For example, if an item is scanned at a packing facility, a flavor profile comprising a flavor score may be forecasted for seven days. During each of the seven days, the item may be scanned again, and the resulting attributes and flavor scores measured against the forecast. The flavor profiler may be refined based on the results of these comparisons.

While some examples provided herein describe produce as an item for which a flavor profile is desired, a flavor profile may be obtained for any type of fresh item, such as dairy, meat, poultry, fish, or any other suitable fresh items.

To enable the flavor profiler to predict flavor and create a flavor profile, forecasting models of flavor scores for an individual item type may be created. In some instances, flavor scores may be forecasted using time series machine learning techniques. The item may be inspected throughout its life cycle, including during transport from growing/producing areas, distribution centers, and/or consumer markets. An item may be inspected, or item data collected, throughout acquisition, production, transportation, storage, and/or sales to identify current item attributes. The collection of data regarding item may be used with machine learning techniques to create forecasting models for each attribute of an item in these examples.

Crunchiness is an example of one attribute associated with an item that may change with time and the environment associated with the item. Thus, knowledge of time and the environmental factors associated with the item may be used when characterizing an item as crunchy. Some items might only be crunchy for a limited amount of time, or under certain environmental conditions. Determining whether an item, characterized as crunchy at a packing location, is still crunchy when it reaches a sales point, may provide more accurate item characteristics to use in promoting sales of the item. The same is true of other attributes such as acidity and sugar levels, for example.

Notably, flavor is different than quality. An item must meet certain regulations before it may be offered to the public. In addition to government regulations, many corporations and/or industries have their own standards. For instance, some companies may only want to provide an item grown or sourced locally. The company may use such selectivity as a differentiating factor when comparing itself to its competitors. This generally relates to the quality of item, not its flavor. However, similar methods and devices may be used to gather information related to both quality and flavor.

Inspecting an item to determine a flavor score is often a time consuming, labor intensive, and technologically challenging endeavor. The logistics involved in gathering the relevant data, making the appropriate decisions based on the data, and implementing the decision, pose a significant challenge for many corporations. Thus, systems and methods that can accurately determine flavor scores, and predict or forecast flavor scores, would provide advantages with managing inventory, increasing sales, and building consumer loyalty.

Referring to the figures, examples of the disclosure enable systems and methods for determining a flavor profile of an item, which in some embodiments, shows flavor scores in relation to shelf life of an item. In some examples, automatic analysis of an item using specific criteria associated with the item is performed by analyzing image data and/or sensor data received from at least one of an imaging component, or one of a set of sensors associated with a mobile computing device. The flavor profiler may also identify defects or unsuitable characteristics associated with an item.

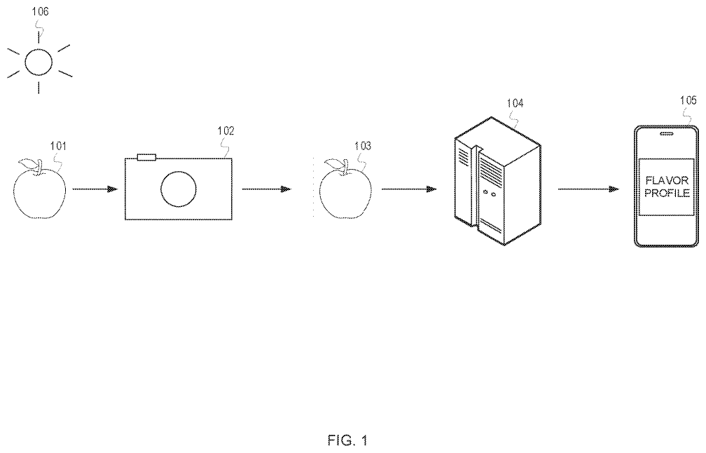

FIG. 1 is a block diagram of embodiments of the systems and methods described herein in accordance with the disclosure. In FIG. 1, an apple 101 is a produce item for which a flavor profile is desired. A diffuse lighting system 106 provides illumination for the apple 101. The diffuse lighting system includes a number of lights. The diffuse lighting system 106 is designed to provide illumination while reducing glare/reflections/etc. Accordingly, in some embodiments, the lights are not directed toward the item. Rather, the lights are designed to provide indirect illumination of the item. For example, the diffuse lighting system 106 can include an enclosure with reflective panels (e.g., placed above, below, and/or around the item). The lights are directed at the reflective panels and the reflective panels reflect light toward the item. In this manner, the diffuse lighting system 106 provide indirect illumination of the item.

An imaging device 102 captures an image of the apple 101. In some cases, the image is a spectral image, while in other cases, the image may be a Red Blue Green (RBG) image of the apple 101. Still other forms of imaging may be used to obtain the necessary information regarding the apple 101. The image 103, which in this example is a spectral image of the apple 101, is output from the imaging device 102 and sent to a computing device 104. In one embodiment, the imaging device 102 is a hyperspectral camera. In such embodiments, the imaging device 102 is configured to capture images over wavelengths ranging from approximately 800 nm to 1800 nm. In a specific embodiment, the imaging device 102 may be configured to a capture a number of images. Some of the image are captured over wavelengths ranging from 400 nm to 1,000 nm and some of the images are captures over wavelengths ranging from 900 nm to 1,700 nm.

The computing device 104 may be a laptop, personal digital assistant, personal computer, server, smartphone or other computing device. The computing device 104 comprises a flavor profiler which is used to forecast the flavor profile of an item. A flavor profiler may take as input the image 103, and/or other forms of data related to the apple 101. A flavor profiler determines attribute scores for the various attributes of the apple 101. A flavor profiler may then determine a flavor score from the various attribute scores. In some cases, the computing device 104 may directly determine a flavor profile of the apple 101 from attribute data and relevant historical data stored in the computing device 104.

The flavor profile may be displayed on a mobile device 105 or other device, in some examples. Alternatively, or additionally, the flavor profile may be accessed via a scannable or machine-readable code, such as a quick response (QR) code or barcode linked to an electronic copy of the flavor profile. In other examples, the computing device 104 and the mobile device 105 may be the same device and may also include the imaging device 102.

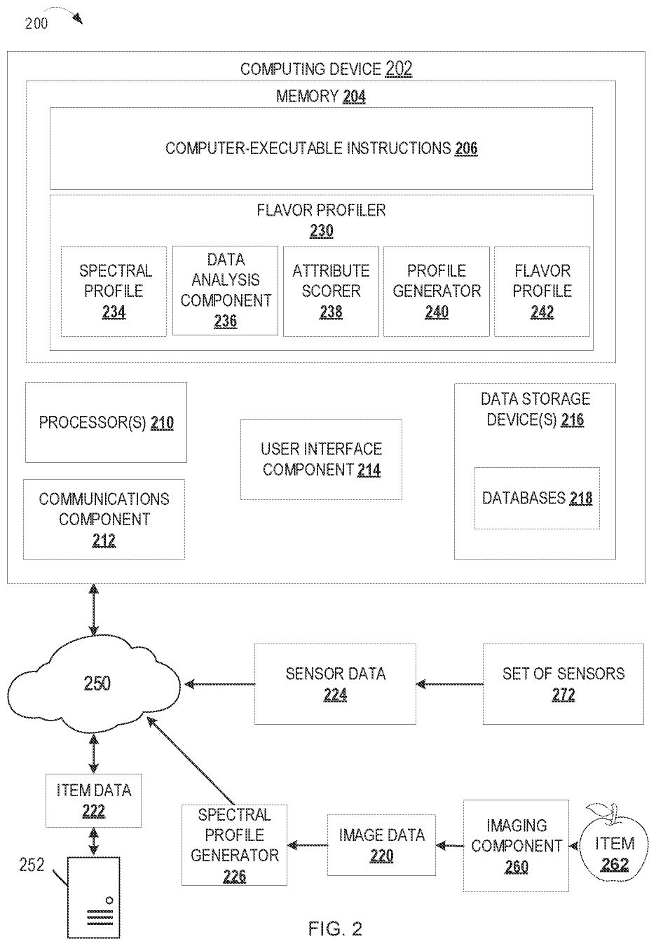

FIG. 2 describes a system for generating a flavor profile for an item. In some instances, the approach described herein may be performed without touching the item.

In one example, an imaging component 260 is used to capture an image of an item 262. The imaging component 260 produces image data 220. The image data 220 may then be sent to a spectral profile generator 226. The spectral profile generator 226 takes image data and converts it into spectral data. The spectral profile generator 226 processes information from across the electromagnetic spectrum to obtain the spectrum for each pixel in an image. Thus, a spectral profile 234 of the item 262 may be obtained using the imaging component 260 and the spectral profile generator 226. The spectral profiling may be performed at an initial scan location and the spectral profile 234 may be used by the flavor profiler 230 to create the flavor profile 242 of the item 262.

In the example of FIG. 2, a computing device 202 is configured to perform embodiments of the systems and methods described herein. The computing device 202 communicates with the imaging component 260 and a set of sensors 272 for gathering image data 220 and sensor data 224 associated with the item 262. Although shown separate from the computing device 202, one of skill in the art will appreciate that the imaging component 260 and the set of sensors 272 may be part of the computing device 202 in other embodiments.

The computing device 202 represents any device executing instructions (e.g., as application programs, operating system functionality, or both) to implement the operations and functionality associated with the computing device 202. The computing device 202 may be a smartphone, mobile telephone, laptop, tablet, personal digital assistant, scanner, computing pad, netbook, gaming device, wearable device, and/or portable media player.

The computing device 202 may also include less-portable devices such as personal computers, servers, kiosks, tabletop devices, and industrial control devices. Additionally, the computing device 202 may represent a group of processing units or other computing devices.

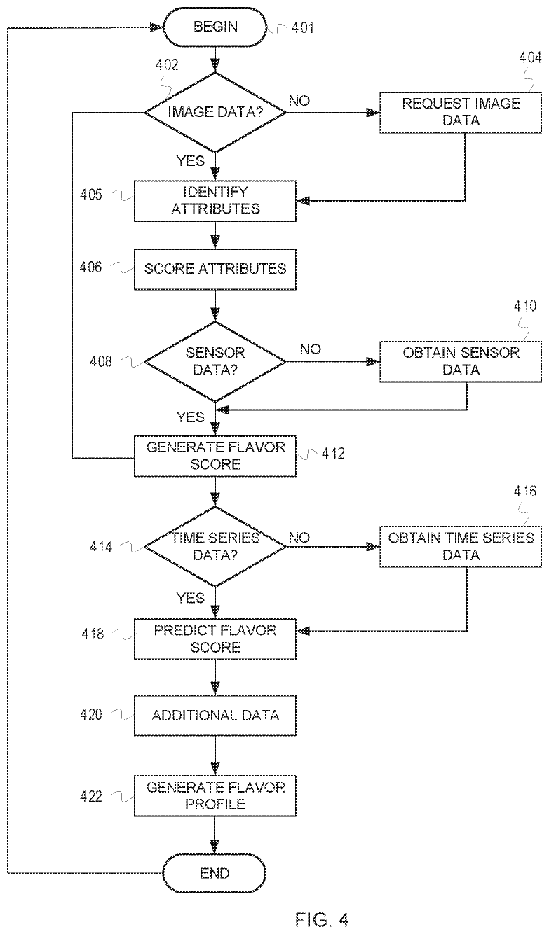

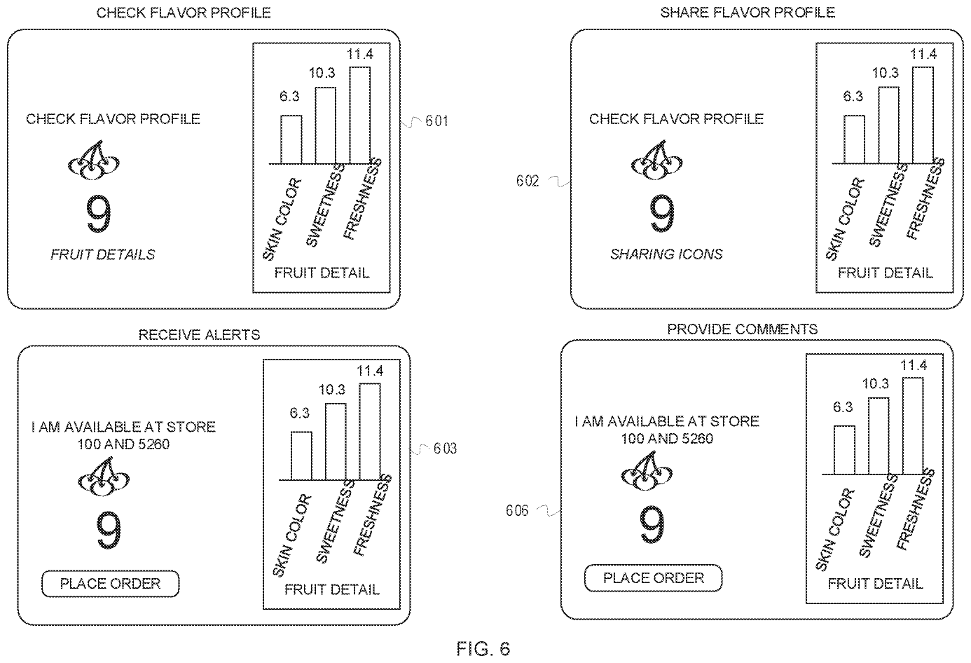

In some examples, the computing device 202 includes one or more processor(s) 210, a memory 204, and a communications component 212. The one or more processor(s) include any quantity of processing units. At least one processor of the one or more processors(s) 210 is programmed to execute computer-executable instructions 206 for implementing the examples herein. The computer-executable instructions 206 may be performed by the processor or by multiple processors within the computing device 202, or performed by a processor external to the computing device 202. In some examples, the processor is programmed to execute instructions such as those illustrated in the figures (e.g., FIG. 4 and FIG. 6).

In some examples, the one or more processor(s) 210 represent an implementation of analog techniques to perform the operations described herein. For example, the operations may be performed by an analog computing device and/or a digital computing device.

The computing device 202 further has one or more computer readable media such as the memory 204. The memory 204 includes any quantity of media associated with or accessible by the computing device 202. The memory 204 may be internal to the computing device 202 (as shown in FIG. 2), external to the computing device 202 (e.g., an external server 252), or both. In some examples, the memory 204 includes read-only memory and/or memory wired into an analog computing device.

The memory 204 stores data, such as one or more applications. An example application is the flavor profiler 230. The applications, when executed by the processor, operate to perform functionality on the computing device 202. The applications may communicate with counterpart applications or services such as web services accessible via a network. For example, the applications may represent downloaded client-side applications that correspond to server-side services executing in a cloud.

The memory 204 further stores one or more computer-executable components. Exemplary components include a user interface component 214 and a communications component 212. In other examples, the memory 204 may include an analysis engine, sensor data, image capture data, and/or processed sensor data.

The imaging component 260 and the set of sensors 272 may send and/or provide the image data 220 and the sensor data 224 to the flavor profiler 230. In some instances, the spectral profile generator 226 may use the image data 220 provide by the imaging component 260 to produce spectral data. In some examples, the image data 220 and the sensor data 224 may include spectral data, temperature data, timestamp data, barometer data, hygrometer data, change of state data, non-IR digital video data, analog video data, still images, light properties or other data generated by one or more sensors. In still other examples, the image data 220 may include IR camera still images, IR camera video images, digital video output, or other image data. For example, the image data 220 may include Moving Pictures Experts Group (MPEG) video output. Thus, the system 200 may obtain image data or sensor data via at least one input device.

In some examples, the flavor profiler 230 obtains the spectral profile 234 directly from the spectral profile generator 226. In other examples, the spectral profile 234 may be obtained via a network 250. The flavor profiler 230 may also obtain sensor data 224 from the set of sensors 272. The set of sensors 272 may correspond to sensors designed to obtain environmental data associated with the item 262, such as temperature, humidity, and pressure for example. In some instances, the image data 220 and the sensor data 224 may be stored in a database 218 or the external server 252 and retrieved by the flavor profiler 230.

The imaging component 260 may be a set of one or more imaging components associated with the computing device 202. The set of imaging components may include one or more spectral analysis devices, one or more image capture devices, one or more scanners, or other such components. A spectral analysis device may also be a hyperspectral imaging device. An image capture device may include an analog camera, a digital camera, an IR camera, or other type of camera. The set of sensors 272 may comprise one or more thermometers, one or more hygrometers, one or more barometers, one or more motion sensors, one or more spectrometers, one or more accelerometers, one or more gas sensors, one or more gyroscopes, one or more motion sensors, one or more Global Positioning System (GPS) sensors, or any other type of sensor. Although shown as separate from the computing device 202, the imaging component 260 and the set of sensors 272 may include one or more imaging components or sensors integrated within the computing device 202 in some examples. In other examples, the imaging component 260 and the set of sensors 272 includes one or more imaging components or sensors external to the computing device 202.

The computing device 202, in some examples, communicates with external devices, cloud infrastructure, and networks via the communications component 212. In FIG. 2, the network 250 is depicted. The external server 252 is communicatively coupled to the network 250. Networks may include, without limitation, a local area network (LAN), such as an Ethernet connection, or a wide area network (WAN), such as the Internet. The network 250 may be a wired network and/or a wireless network, such as WI-FI. The set of sensors 272 may transmit the sensor data 224 via the network 250 in response to a request from the flavor profiler 230. In other examples, the set of sensors 272 automatically transmits the sensor data 224 to the flavor profiler 230 in real, or near-real, time as the sensor data 224 is generated. In still other examples, the sensor data 224 is sent at regular or predetermined intervals or in response to a predetermined event.

The communications component 212 may include a network interface card and/or computer-executable instructions (e.g., a driver) for operating the network interface card. Communication between the computing device 202 and other devices may occur using any protocol or mechanism over any wired or wireless connection. In some examples, the communications interface is operable with short range communication technologies such as by using near-field communication (NFC) tags.

In some examples, the flavor profiler 230 may generate an alert and output notification of the alert to one or more user interfaces via user interface component 214. Alerts may inform a user of the availability of a particular item with a particular flavor score. Other alerts may be to inform personnel of conditions associated with an item, such as a sudden rise in temperature in a container used for shipping an item. A user interface may be a display device that presents the alert to a user or a speaker that generates an audible sound which the user can hear. Other user interfaces are also possible. Examples of other interfaces are command line prompts, graphical user interfaces, augmented reality, tactile and feedback interfaces, digital menus, and the like.

The user interface component 214 may include a graphics card for displaying data and alerts to one or more users, as well as receiving data from one or more users. The user interface component 214 may also include computer-executable instructions (e.g., a driver) for operating the graphics card. Further, the user interface component 214 may include a display (e.g., a touch screen display device or natural user interface) and/or computer-executable instructions (e.g., a driver) for operating the display device. The user interface component 214 may also include one or more of speakers, a sound card, a camera, a microphone, a vibration motor, one or more accelerometers, a BLUETOOTH brand communication module, global positioning system (GPS) hardware, and a photoreceptive light sensor to provide data to the user or receive data from the user. For example, the user may input commands or manipulate data by moving the computing device in a particular manner.

The flavor profiler 230 in this example is stored within the memory 204. In other examples, the flavor profiler 230 may be stored within one or more database(s) 218 and/or within one or more data storage devices(s) 216 and retrieved for use. The database 218 may store information regarding items and time data associated with the items which may be used by the flavor profiler 230 to determine the flavor profile 242 for item 262.

In other examples, the data storage may also be implemented externally to the computing device 202 such as in the external server 252 and accessed via the network 250. The data storage device(s) may include rotational storage, such as a disk and/or non-rotational storage media, such as a solid-state drive (SSD) or flash memory. In some non-limiting examples, the data storage device(s) provide a shared data store accessible by two or more hosts in a cluster. For example, the data storage device may include a hard disk, a redundant array of independent disks (RAID), a flash memory drive, a storage area network (SAN), or other data storage device.

The flavor profiler 230 predicts the flavor of an item over the shelf life of the item. The flavor profiler 230 obtains spectral image data from spectral profile generator 226, and the sensor data 224 from the set of sensors 272. A data analysis component 236 identifies attributes of the item 262 based on the obtained spectral profile 234. An attribute scorer 238 generates a score, or attribute value, for each attribute identified by the data analysis component 236. The attribute score may indicate a level of the attribute. For example, the attribute score may be based on a scale from 1 to 10. If, for example, the attribute being measured is ripeness, a score of 5 may indicate that the item is ripe, while a score of 0 or 10 might indicate the item is either not ripe or overripe, respectively. A profile generator 240 uses the attribute scores from the attribute scorer 238 and additional data, in some cases item data 222, to predict or forecast a flavor score. A prediction of a flavor score shows how the flavor of an item changes over time. The prediction is performed using various algorithms and stored data related to the item 262. The flavor score is then used together with attribute data and attribute scores to create the flavor profile 242 for the item 262. The flavor profile 242 may also comprise additional information, such as from where the item originated, how long the item has been in transport, etc.

The item data 222 may be obtained by collecting and analyzing data on the rate at which an attribute, and thus the attribute score of the attribute, deteriorates over time as an item progresses towards the end of its shelf life. The item data 222 may include data about an item over varying time segments under different conditions. For example, the item data 222 may comprise data indicating the crispness of a particular type of apple over a day, weeks, months, etc. These data may be used with other data, such as origination data, and transportation time, to predict how crisp a certain apple may be when it reaches its final destination. Other forms of item data may also be possible. The item data 222 may be stored in the databases 218 or the external server 252. The item data 222 may also be computed from the sensor data 224 and environmental data, along with historical data stored in the databases 218 and/or the external server 252. It should be appreciated that the flavor profiles may be generated multiple times for an item or updated depending on the circumstances. For example, a piece of fruit that undergoes a long transportation process may have its flavor profile computed several times over the course of the supply chain and transportation journey.

In some embodiments, a flavor profile may be made available to a consumer via a mobile application, a website, or other electronic means. The flavor profile comprises at least a flavor score. Notably, with regards to the flavor score, higher is not necessarily better. Such scores simply convey information. Customers may choose a desired flavor score (e.g., a customer may prefer tart or sweet for an apple). A lower score may indicate that that item 262 is not ripe yet (score may increase in a flavor profile), is overripe (score may decrease in a flavor profile), or the firmness of fruit (some customers like more firm, some like less firm and more juicy), among other possibilities.

Any attribute of the item 262 that may be derived from the image data 220 or sensed by a sensor, directly or indirectly, or calculated based on the sensor data 224, may be a basis for a flavor profile of the item 262. Different criteria may be used for different items. Example attributes may include, without limitation, sweetness, crispiness, firmness, tartness, sourness, acidity, color, and any other suitable attribute.

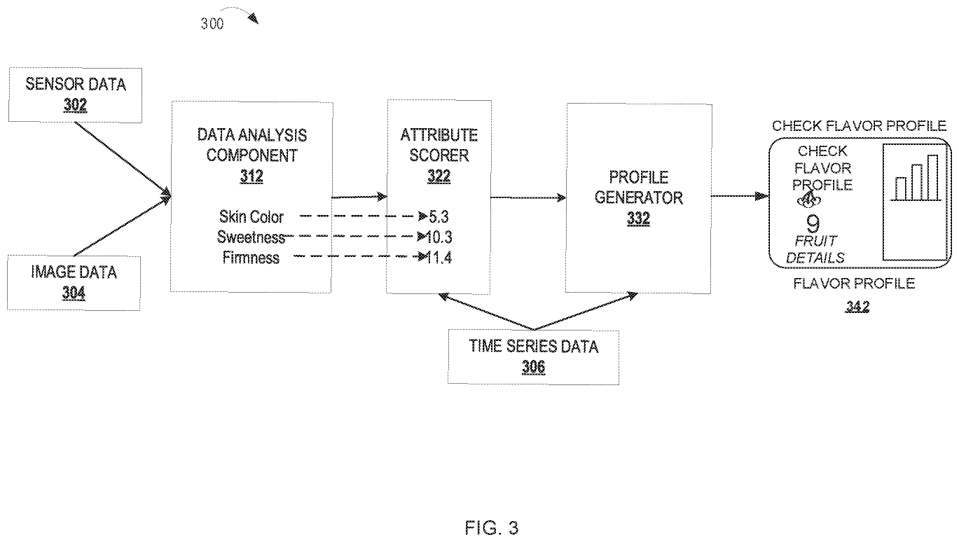

FIG. 3 describes an example embodiment of a flavor profiler 300, such as the flavor profiler 230 depicted in FIG. 2. A data analysis component 312 receives sensor data 302 and image data 304. In this example, the image data 304 includes spectral data regarding an item. Spectral data may be used to create a spectral profile, in some examples, and include one or more characteristics of the item obtained from a spectral imaging device. The image data 304 may include any data pertaining to the item used to determine a score for an attribute of the item, such as, without limitation, color, weight, quality, condition, defects, or any other suitable characteristic, for example.

Sensor data 302 is any other data related to an item, which may be used in creating a flavor profile of an item. Such data may include origin data, growth data, weather data, transportation time, transportation method, location data, etc. The sensor data 302 may be obtained from sensors.

The data analysis component 312 determines a set of attributes pertaining to the item. In this non-limiting example, the data analysis component 312 determines three attributes associated with the item: 1) skin color, 2) sweetness, and 3) firmness. However, other attributes, such as acidity, crunchiness, ripeness, firmness, and sugar levels, may also be identified. Attributes may be identified or inferred from image data, a spectral profile, or other identifying characteristics. For example, an attribute such as skin color may be directly identified in the image data 304, which in some cases may be a spectral image. In other cases, such as for an attribute such as firmness, the image data 304 may be combined with algorithms and data corresponding to the item, and the attribute may be inferred or determined by such processing.

In other embodiments, different attributes may be determined, such as crunchiness, acidity, tartness, color, freshness, aroma, etc. The attributes identified by the data analysis component are then scored by an attribute scorer 322.

Time series data 306 may comprise historical or machine learned data regarding an item. For example, the time series data 306 may comprise data showing the rate at which an item similar to the item loses its firmness over several days. The time series data 306 may be combined with analysis methods in order to extract statistics and other characteristics of the data. Forecasting may then be performed by a profile generator 332 to predict future values based on previously computed or observed value.

Each attribute may, in some instances, be assigned a separate score. In this example, each attribute identified by the data analysis component 312 has been initially scored. In some instances, a convolution neural network (CNN) may be used to score the attribute. A CNN is an artificial neural network which applies a convolution operation to an input and passes the result to the next layer. The convolution emulates the response of neurons. In this example, a CNN may be used to analyze the spectral profile or image data, extract attributes, and score the attributes. In this example, the attribute of skin color was scored 5.3, the attribute of sweetness was scored 10.3, and the attribute of firmness was scored 11.4. In other instances, the attribute scorer may simply determine a combined score. In other instances, attribute scorer may grade the individual attributes or the item as a whole. It will be appreciated by one of skill in the art that other methods of scoring the item for forecasting shelf life is also possible. Additionally, a flavor score may be calculated based on a first attribute score or value and a second attribute score or value of the item.

The attribute scores are then sent to profile generator 332. The attribute scores may be used to determine a flavor score. In some instances, the time series data 306 may be used in forecasting a change in a flavor score. The predicted or forecasted flavor score may be used as the basis for the flavor profile 342. In other instances, the flavor profile 342 may be based on the individual attribute scores which have been forecasted. In this example, the item has a flavor score of 9. In this example, the flavor score of 9 may be a weighted average of its skin color, firmness, and sweetness. The profile generator 332 may include a customized algorithm, a set of rules, or any other computer implemented method to determine the flavor profile based on the data. The profile generator 332 may be specific to a particular item, a set of items, a type of item, or a classification of items. Items may be sampled individually or in batches. In some embodiments, the flavor profile 342 may be generated locally on a computing device used to provide the flavor profile to a user, while in other aspects, the flavor profile 342 may be computed using another device, network server, or cloud infrastructure. Additionally, the profile generator 332 may be further customized in any given manner and various combinations of data may be used to determine the flavor profile 342. It should be appreciated by one of skill in the art that the profile generator 332 may be customized in a variety of ways to address the needs of specific stores, institutions, inspections, personnel, agencies, management, or the like.

FIG. 4 is an exemplary flow chart of a process for generating a flavor profile for an item. The process shown in FIG. 4 may be performed by a flavor profiler, such as the flavor profiler 230 of FIG. 2, executing on a computing device. Further, execution of the operations illustrated in FIG. 4 is not limited to a flavor profiler. FIG. 4 is one example of a process for determining a flavor profile. One of skill in the art will recognize that other processes and variations may also be used instead of or together with the processes described in and related to FIG. 4. One or more computer-readable storage media storing computer-readable instructions may execute to cause at least one processor to implement the operations illustrated in FIG. 4.

The process beings at operation 401. The process may be triggered by any number of events such as receipt of image data of an item. In some cases, the process may be manually triggered, for example, at a packaging and/or packing facility of the item. In other cases, it may be automated such as upon scanning of a received item from a grower. In still other cases, it may be based upon periodic updates, which may happen when a package is in transit. A determination is made as to whether image data has been received from one or more sources at operation 402. Image data may be a spectral profile in some instances received from a spectral imaging component. A received spectral profile may comprise a number of identified attributes. For example, a hyperspectral imaging device may be used to obtain relevant data regarding an item, such as color or temperature spectrum of the item. This data may then be used to determine attributes of the item, such as sweetness, or crunchiness. The attributes may then form the basis of a spectral profile. The image data in operation 402 may comprise the spectral profile.

Other types of image data for operation 402, and other sources of data, are also possible such as web servers, web services, internet servers, remote databases, and the like. Image data may be obtained from sources other than an imaging component. In some embodiments, the sources of image or spectral data may be external to the computing device, such as a remote scanner or spectral data stored in cloud infrastructure. In other aspects, the sources may be local to the device such as an on-board spectrometer or added to the device using a USB drive or similar device.

If the image data has not been received, image data is requested at operation 404 by a flavor profiler, such as flavor profiler 230 in FIG. 2 for example, or another similar component. In one example, the image data is spectral data which may be gathered prior to packaging at a pack center. In other examples, the image data may be gathered at other times, such as during transit or during inspection. At operation 405, attributes are identified from the image data, and at operation 406, the attributes are scored.

At operation 408, a determination is made as to whether sensor data is needed from one or more sources. This determination may be based on what attributes have been determined. For example, if one of the attributes is firmness, then sensor data related to temperature may be helpful in predicting a change in the attribute score. Sensor data may be obtained from a set of sensors, or as with image data, sensor data may be obtained from remote scanners, web servers, web services, interne servers, remote databases, and the like. In other aspects, the sensor data sources may be local to the device or added to the device using a USB drive or similar device. At operation 410, sensor data may be requested by flavor profiler 230.

At operation 412, a flavor score is generated. The flavor score may be determined based on the scored attributes. In some instances, the flavor score is a weighted averaging of the scored attributes. In other cases, different algorithms may be used to determine the flavor score. Furthermore, the algorithms may be customized by a user.

Although in the current example the flavor score is shown to be determined prior to forecasting any scores, it should be appreciated by one of skill in the art that different methodologies are also contemplated. For instance, the attributes may be scored, and the attribute scores themselves may be forecasted. In this case, the flavor score is a predicted flavor score, which may have been computed from the forecasted attribute scores.

At operation 414, a determination is made as to whether time series data has been obtained. If time series data has not been obtained, then at operation 416 it is obtained. Time series data is used to forecast scores. In this example, time series data is used with attribute scores to forecast the flavor score at operation 418. In other examples, time series data may be used to forecast individual attribute scores, which then, in turn, is used to determine a flavor score at a given point in time.

At operation 420, additional data may be requested from a set of sensors, such as set of sensors 272 in FIG. 2. Additional data in some examples include data such as weight or density of an item, or data from third party applications or from transportation databases. In other cases, additional data may be thresholds or the like associated with item or other aspects of the process. For example, a threshold may be a minimum weight of an item for it be sold in a store. In some embodiments, the sources of additional data may be external to the computing device, such as networked servers or drives. As with image data, additional data may be from remote scanners, web servers, web services, interne servers, remote databases, and the like. In other aspects, the additional data sources may be local to the device or added to the device using a USB drive or similar device.

At operation 422, a flavor profile is generated based on the attribute scores, the predicted flavor score, and additional data, such as shown by a screenshot of the first user interface 601 FIG. 6. In this example, each attribute has a score presented on the flavor profile. Additionally, a flavor score of the item is also presented. Finally, additional data such as origin information of the item, is presented to the user. It should be appreciated that only a subset of the listed data may be needed to generate a flavor score in some instances. Flavor profiles may be specific to a particular item or type of item. The process of generating a flavor score may be repeated as necessary. For instance, the additional data may comprise transportation data associated with transportation of the item post-packing, and the flavor profile may be updated with a newly generated flavor score based at least in part on the received transportation data.

Although the process described in FIG. 4 envisions sensed or manually input data as spectral data and/or image data, other sources of data or any of the like may be used as item data. While the operations illustrated in FIG. 4 are performed by a computing device, aspects of the disclosure contemplate performance of the operations by other entities or multiple entities working in concert or independently. For example, a cloud service may perform one or more of the operations.

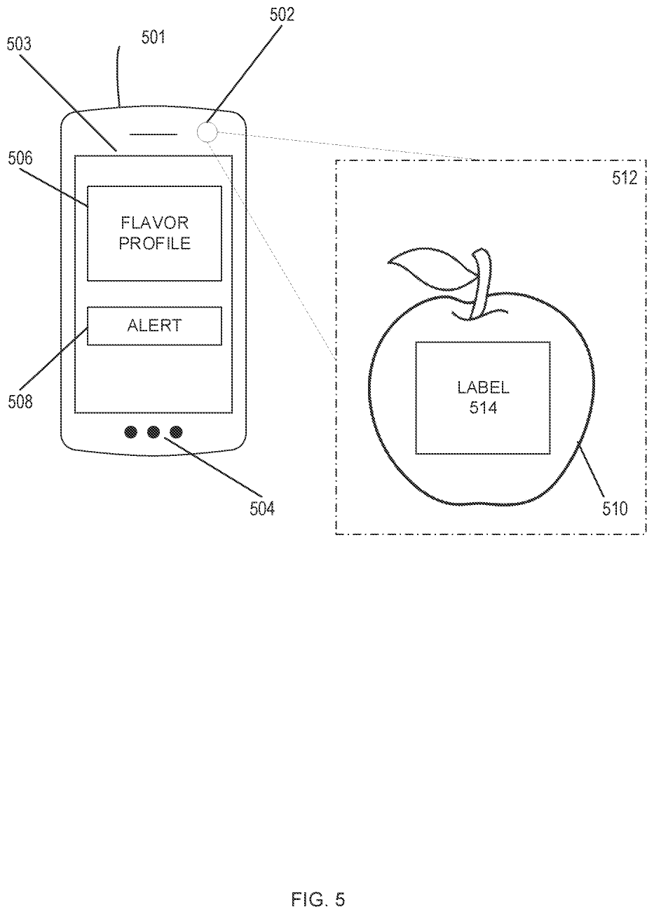

FIG. 5 is an exemplary illustration of a device 501 retrieving information about an item 510. The device 501 comprises an image sensor 502 and a touchscreen 503. Additionally, the device 501 may comprise other sensors represented by reference numeral 504. The device 501 uses the image sensor 502 to obtain sensor data regarding the item 510. In some instances, the item 510 may be in a package 512, or other protective material. The package 512 may further comprise a label 514. The image sensor 502 may scan the label to obtain data related to the item 510, such as a flavor profile 506. The flavor profile 506 may be the flavor score and the forecast displayed separately, or the flavor score forecasted over a given time period, or any permutation of the above. In other embodiments, sensor data may be analyzed by the flavor profiler (not shown) on device 501 to generate the flavor profile 506.

An alert 508 may be an automated alert sent to a user when a desired flavor score is available. For example, a user may input desired flavor scores for specific items. The system may dynamically analyze generated flavor profiles for such items, and upon detecting the desired flavor score in a flavor profile of such an item, generate an alert directed to a user interface associated with the user. Detailed information for the location of the item (store) and expected date of availability would also be provided. Other alerts may also be possible.

In some instances, the flavor profiler may reside on a remote computing device or remote server such as an external server (not shown). In some instances, the flavor profile 506 may be presented to a user via the touchscreen 503. In other instances, the flavor profile 506 may be transmitted for further analysis, such as to an external server, such as to determine whether such a profile comprises a desired flavor score. In addition to the flavor profile 506, any other available data associated with the item 510 may be retrieved such as the origin of an item 510, the harvest date, the remaining shelf life, etc. Other forms of additional data may also be retrieved, such as information related to health benefits or concerns, availability, facts, supply chain information and the like.

Although shown with regards to a device with the touchscreen 503, other implementations are also possible. Additionally, discrete aspects may be implemented in other methods. For example, the flavor score may be displayed on a smart display associated with the item 510 with which a customer may interact. Alternatively, an item display may use a QR code which may be scanned by a user device and the flavor profile 506 may be retrieved for display on the device 501.

FIG. 6 shows various examples of flavor profiles and uses thereof. FIG. 6 shows only some examples of potential outputs of a flavor profiler such as flavor profiler 230 of FIG. 2. One of skill in the art will appreciate that other outputs are possible. A first user interface 601 shows a flavor profile associated with an example item of cherries. The flavor score is indicated as a numeric score of 9. The first user interface 601 further shows additional data which may be presented to a user which may be part of the flavor profile such as the origin of the cherries, the harvest date, and the remaining shelf life. Other exemplary additional data may include identified compounds of cherries that minimize the risk of several diseases, and specifically calls out those compounds and disease. For example, some consumers may want information regarding health benefits, while in other cases, they may want information regarding whether the item is locally sourced. It should be appreciated that there is no limit to the type of information that may be retrieved and presented.

In some instances, the flavor profile may comprise only the flavor score, or a forecasted attribute score. In the first user interface 601, each of the attributes are equally weighted which results in a flavor score of 9. In other embodiments, the attributes may be weighted differently. For example, the flavor profile may further comprise information regarding origination of the item, or information regarding transportation of the item or information regarding quantity of the item available. Other algorithms may be used to determine a flavor score or forecast any determined score. Furthermore, the algorithms may be customized based on user input.

A second user interface 602 depicts options for sharing a flavor profile using social media. For example, when a customer scans the label on a piece of fruit, information related to price, store location and flavor profile may be populated on the user interface. Image matching may also be performed to determine the type of item. This information may then be shared via a number of different social media options.

A third user interface 603 depicts a user interface for subscribing to a desired flavor score or profile and receiving alerts based on the detected flavor score or profile. A consumer may use a flavor profiler application to select item(s) of interest, set a preferred store location and then subscribe to receive alerts when a desired flavor score or profile for the selected item becomes available at the selected location. In addition, he/she may also opt-in to automatically have a desired flavor profile of a selected item ready for store pick up or delivered at their home as and when available or on a given day or set interval of time. The above example may also be accomplished using digital assistants as an added API to a store application or similar program. The store application may recognize the user's location, and advise on the availability of a desired flavor profile, or advise of all possible stores in which the flavor profile is available within a specified radius or area (e.g., 5 miles, 10 miles, etc.).

A fourth user interface 606 shows an example of a flavor profiler application providing a mechanism for a user to provide comments. Comments may be in the form of questions sent to the store or may be notations for the user to use for themselves (“remember to buy pie crust for cherries”). In some instances, the customer may able to share feedback with a grower and also ask questions of a grower. Other options may allow a user to learn how an item was grown, including irrigation and cultivation activities. It may also allow a user to understand what types of fertilizers were used, or learn about how items travelled from a grower, entered the supply chain and/or arrived at stores, for example.

In some instances, block chain technology may be used to trace how an item was grown and information from the service is also made available to study and learn the above details.

### Additional Examples

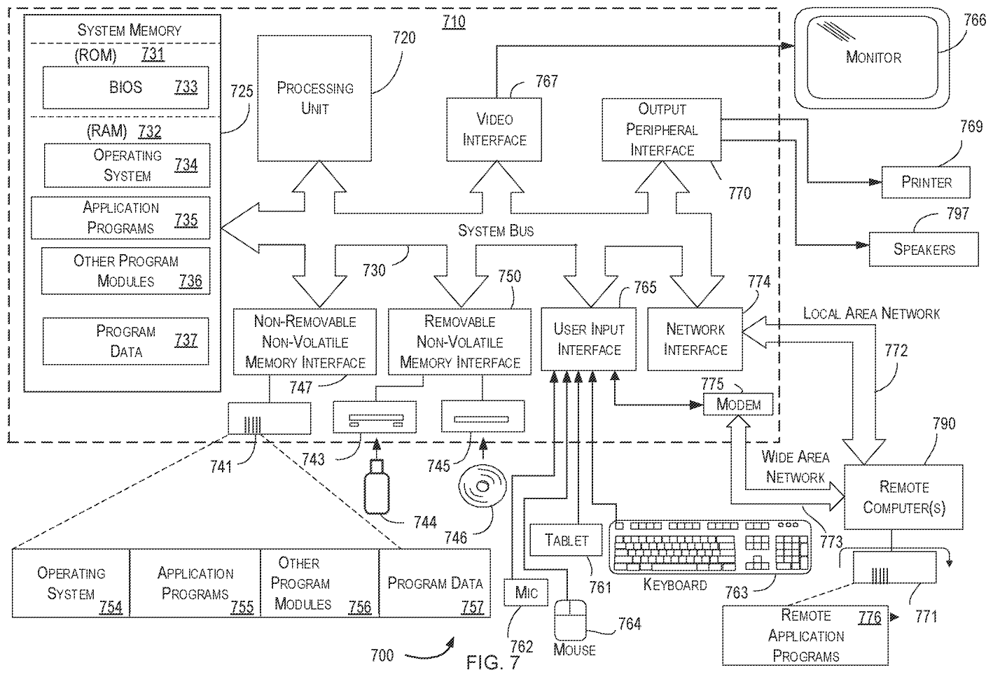

At least a portion of the functionality of the various elements in FIG. 1, FIG. 2, FIG. 3, FIG. 4, and FIG. 5 may be performed by other elements in FIG. 1, FIG. 2, FIG. 3, and FIG. 7, or an entity (e.g., processor, web service, server, application program, computing device, etc.) not shown in FIG. 1, FIG. 2, FIG. 3, FIG. 4, FIG. 5, FIG. 6 and FIG. 7

In some examples, the operations illustrated in FIG. 3 and FIG. 4, and may be implemented as software instructions encoded on a computer readable medium, in hardware programmed or designed to perform the operations, or both. For example, aspects of the disclosure may be implemented as a system on a chip or other circuitry including a plurality of interconnected, electrically conductive elements. Operations may be performed on a single device, or multiple devices communicatively coupled together. Aspects of the disclosure may be performed at one location or different locations communicatively coupled together. Additionally, results of some operations may be sent as copies to different devices. For instance, a network server may be used to generate a freshness indicator or shelf life of an item. The generated freshness indicator or shelf life of an item may then be sent to a plurality of computing devices. Additionally, aspects of the disclosure such as notifications and alerts may be sent to a single device, multiple devices or used by other applications or programs.

Spectral data, time data, item data, environmental data, or any other data, may be obtained from a variety of sources, such as, but without limitation, spectral instruments, historical data collected by companies, trade groups, or other such organizations. Data associated with an item may be manually input by users or those with knowledge of the item. Information gathered by sensors may be used to not only to determine item's flavor profile but also whether an item is acceptable as is. Other determinations may include expiration date, use-by dates, sell-by dates etc. For instance, bananas stored in special rooms may be treated with temperature and/or gas to ripen or delay ripening, which may help determine the flavor score and forecast. Ripeness would assist in forecasting sell-by dates.

Communication and storage may be accomplished by a variety of devices and through a variety of means. Communication may be over wireless networks such as BLUETOOTH or Wi-Fi or over wired networks such as ethernet and phone lines. Communication may be direct through devices, through corporate intranets, the internet, cloud-based services or the like. Similarly, storage of data and components may be on computing devices, local servers, network servers, remote servers, cloud applications and devices, mobile devices, kiosks, etc.

In some embodiments, customers can share flavor profiles with family, friends, contacts, etc. via, for example social media. In such embodiment, a customer can share a flavor profile for an item that he or she enjoyed. The customer, as well as his or her contact on the social media platform can sign up to receive alerts from a retailer when an item matching that flavor profile arrives at the retailer.

While the aspects of the disclosure have been described in terms of various examples with their associated operations, a person skilled in the art would appreciate that a combination of operations from any number of different examples is also within scope of the aspects of the disclosure.

Alternatively, or in addition to the other examples described herein, examples include any combination of the following:
A computing system for determining a flavor profile of an item, the computing system comprising: a memory device storing computer-executable instructions; and a processor configured to execute the computer-executable instructions to: obtain a spectral profile associated with the item; identify at least one attribute value for at least one attribute of the item based on the received spectral profile; determine a flavor score for the item based on the at least one attribute value; obtain time series data associated with the item corresponding to a number of days; calculate a predicted flavor score for the item relative to the number of days based on the received time series data; and generate a flavor profile for the item based at least on the predicted flavor score.
wherein the at least one attribute comprises at least one of acidity, crunchiness, ripeness, firmness, and sugar levels.
wherein the processor is further configured to execute the computer-executable instructions to generate the flavor profile for each day of the number of days.
wherein the processor is further configured to execute the computer-executable instructions to obtain sensor data via at least one input device.
wherein the processor is further configured to execute the computer-executable instructions to calculate the predicted flavor score based on the identified at least one attribute value and at least a second attribute value of the item.
wherein the processor is further configured to execute the computer-executable instructions to update the flavor profile at predetermined intervals.
wherein the flavor profile is accessed via a barcode or quick reference (QR) code on a packing material associated with the item.
wherein the processor is further configured to execute the computer-executable instructions to receive transportation data associated with transportation of the item post-packing via at least one input device, and to update the flavor profile based at least in part on the received transportation data.
wherein the flavor profile further comprises information regarding origination of the item.
wherein the flavor profile further comprises information regarding transportation of the item.
wherein the flavor profile further comprises information regarding quantity of the item available.
wherein the processor is further configured to execute the computer-executable instructions to determine at least one of the following based on the flavor profile: priority of placement of the item in a store from an inventory of an item type associated with the item, selection of a distribution center for the item, pricing of the item, inspection criteria for the item; safety stock number of the item at the store, shelf life of the item, quality metrics associated with different suppliers of the item type, and impact of weather on the quality of the item type.
wherein the flavor profile is accessed via a bar code or QR code on a packing material associated with the item.
wherein the flavor profile further comprises information regarding origination of the item.
wherein the processor is further configured to execute the computer-executable instructions to determine at least one of the following based on the flavor profile: priority of placement of the item in a store from an inventory of an item type associated with the item, selection of a distribution center for the item, pricing of the item, inspection criteria for the item; safety stock number of the item at the store, shelf life of the item, quality metrics associated with different suppliers of the item type, and impact of weather on the quality of the item type.
wherein the processor is further configured to execute the computer-executable instructions to generate the flavor profile for each day of the number of days.

The term “Wi-Fi” as used herein refers, in some examples, to a wireless local area network using high frequency radio signals for the transmission of data. The term “BLUETOOTH” as used herein refers, in some examples, to a wireless technology standard for exchanging data over short distances using short wavelength radio transmission. The term “cellular” as used herein refers, in some examples, to a wireless communication system using short-range radio stations that, when joined together, enable the transmission of data over a wide geographic area. The term “NFC” as used herein refers, in some examples, to a short-range high frequency wireless communication technology for the exchange of data over short distances.

### Example Operating Environment

FIG. 7 is a block diagram illustrating an example operating environment 700 for a computing device. The computing environment is only one example of a suitable computing environment and is not intended to suggest any limitation as to the scope of use or functionality of the disclosure. Neither should the computing environment be interpreted as having any dependency or requirement relating to any one or combination of components illustrated in the example operating environment 700.

The disclosure is operational with numerous other general purpose or special purpose computing system environments or configurations. Examples of well-known computing systems, environments, and/or configurations that may be suitable for use with the disclosure include, but are not limited to: personal computers, desktop computers, laptop computers, tablet devices, netbooks, handheld devices, mobile telephones, wearables, gaming devices, portable media players, server computers, kiosks, set top boxes, tabletop devices, multiprocessor systems, microprocessor-based systems, programmable consumer electronics, network PCs, minicomputers, mainframe computers, distributed computing environments that include any of the above systems or devices, and the like.

The disclosure may be described in the general context of computer-executable instructions, such as program modules, being executed by a computer. Generally, program modules include routines, programs, objects, components, data structures, and so forth, which perform particular tasks or implement particular abstract data types. The disclosure may also be practiced in distributed computing environments where tasks are performed by remote processing devices that are linked through a communications network. In a distributed computing environment, program modules may be located in local and/or remote computer storage media including memory storage devices and/or computer storage devices. As used herein, computer storage devices refer to hardware devices.

With reference to FIG. 7, an example system for implementing various aspects of the disclosure may include a general-purpose computing device in the form of a computer 710. Components of the computer 710 may include, but are not limited to, a processing unit 720, a system memory 725, and a system bus 730 that communicatively couples various system components including the system memory to the processing unit 720. The system bus 730 may be any of several types of bus structures including a memory bus or memory controller, a peripheral bus, and a local bus using any of a variety of bus architectures. By way of example, and not limitation, such architectures include Industry Standard Architecture (ISA) bus, Micro Channel Architecture (MCA) bus, Enhanced ISA (EISA) bus, Video Electronics Standards Association (VESA) local bus, and Peripheral Component Interconnect (PCI) bus also known as Mezzanine bus.

The computer 710 typically includes a variety of computer-readable media. Computer-readable media may be any available media that may be accessed by the computer 710 and includes both volatile and nonvolatile media, and removable and non-removable media. By way of example, and not limitation, computer-readable media may comprise computer storage media and communication media. Computer storage media includes volatile and nonvolatile, removable and non-removable media implemented in any method or technology for storage of information such as computer-readable instructions, data structures, program modules or the like. A read only memory (ROM) 731 and a random-access memory (RAM) 732 are examples of computer storage media. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other magnetic storage devices, or any other medium which may be used to store the desired information and which may be accessed by the computer 710. Computer storage media does not, however, include propagated signals. Rather, computer storage media excludes propagated signals. Any such computer storage media may be part of computer 710.

Communication media typically embodies computer-readable instructions, data structures, program modules or the like in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media.

The system memory 725 includes computer storage media in the form of volatile and/or nonvolatile memory such as ROM 731 and RAM 732. A basic input/output system 733 (BIOS), containing the basic routines that help to transfer information between elements within computer 710, such as during start-up, is typically stored in ROM 731. RAM 732 typically contains data and/or program modules that are immediately accessible to and/or presently being operated on by processing unit 720. By way of example, and not limitation, FIG. 7 illustrates operating system 734, application programs, such as application programs 735 (e.g., appliance management environment), other program modules 736 and program data 737.

The computer 710 may also include other removable/non-removable, volatile/nonvolatile computer storage media. By way of example only, FIG. 7 illustrates a hard disk drive 741 that reads from or writes to non-removable, nonvolatile magnetic media, a universal serial bus (USB) port 743 that provides for reads from or writes to a removable, nonvolatile memory 744, and an optical disk drive 745 that reads from or writes to a removable, nonvolatile optical disk 746 such as a CD ROM or other optical media. Other removable/non-removable, volatile/nonvolatile computer storage media that may be used in the example operating environment include, but are not limited to, magnetic tape cassettes, flash memory cards, digital versatile disks, digital video tape, solid state RAM, solid state ROM, and the like. The hard disk drive 741 is typically connected to the system bus 730 through a non-removable memory interface such as interface 747, and USB port 743 and optical disk drive 745 are typically connected to the system bus 730 by a removable memory interface, such as interface 750.

The drives and their associated computer storage media, described above and illustrated in FIG. 7, provide storage of computer-readable instructions, data structures, program modules and other data for the computer 710. In FIG. 7, for example, hard disk drive 741 is illustrated as storing operating system 754, application programs 735 (e.g., an appliance management environment), other program modules 756 and program data 757. Note that these components may either be the same as or different from operating system 734, application programs 735, other program modules 736, and program data 737. Operating system 754, application programs 735, other program modules 756, and program data 757 are given different numbers herein to illustrate that, at a minimum, they are different copies.

A user may enter commands and information into the computer 710 through input devices such as a tablet, or electronic digitizer, 761, a microphone 762, a keyboard 763 and pointing device 764, commonly referred to as mouse, trackball or touch pad. Other input devices not shown in FIG. 7 may include a joystick, game pad, digital camera, scanner, or the like. These and other input devices are often connected to the processing unit 720 through a user input interface 760 that is coupled to the system bus, but may be connected by other interface and bus structures, such as a parallel port, game port or a universal serial bus (USB). A monitor 766 or other type of display device is also connected to the system bus 730 via an interface, such as a video interface 767. The monitor 766 may also be integrated with a touchscreen panel or the like. Note that the monitor and/or touchscreen panel may be physically coupled to a housing in which the computer 710 is incorporated, such as in a tablet device. In addition, computers such as the computer 710 may also include other peripheral output devices such as speakers 797 and printer 769, which may be connected through an output peripheral interface 770 or the like.

The computer 710 may operate in a networked environment using logical connections to one or more remote computers, such as a remote computer 790. The remote computer 790 may be a personal computer, a server, a router, a network PC, a peer device or other common network node, and typically includes many or all of the elements described above relative to the computer 710, although only a memory device 771 has been illustrated in FIG. 7. The logical connections depicted in FIG. 7 include one or more local area networks (LAN) 772 and one or more wide area networks (WAN) 773, but may also include other networks. Such networking environments are commonplace in offices, enterprise-wide computer networks, intranets and the Internet.

While the disclosure is susceptible to various modifications and alternative constructions, certain illustrated examples thereof are shown in the drawings and have been described above in detail. It should be understood, however, that there is no intention to limit the disclosure to the specific forms disclosed, but on the contrary, the intention is to cover all modifications, alternative constructions, and equivalents falling within the spirit and scope of the disclosure. Exemplary computer readable media include flash memory drives, digital versatile discs (DVDs), compact discs (CDs), floppy disks, and tape cassettes. By way of example and not limitation, computer readable media comprise computer storage media and communication media. Computer storage media include volatile and nonvolatile, removable and non-removable media implemented in any method or technology for storage of information such as computer readable instructions, data structures, program modules and the like. Computer storage media are tangible and mutually exclusive to communication media. Computer storage media are implemented in hardware and exclude carrier waves and propagated signals. Computer storage media for purposes of this disclosure are not signals per se. Exemplary computer storage media include hard disks, flash drives, and other solid-state memory. In contrast, communication media typically embody computer readable instructions, data structures, program modules, or the like, in a modulated data signal such as a carrier wave or other transport mechanism and include any information delivery media.

Although described in connection with an exemplary computing system environment, examples of the disclosure are capable of implementation with numerous other general purpose or special purpose computing system environments, configurations, or devices.

Examples of well-known computing systems, environments, and/or configurations that may be suitable for use with aspects of the disclosure include, but are not limited to, mobile computing devices, personal computers, server computers, hand-held or laptop devices, multiprocessor systems, gaming consoles, microprocessor-based systems, programmable consumer electronics, mobile telephones, mobile computing and/or communication devices in wearable or accessory form factors (e.g., watches, glasses, headsets, or earphones), network PCs, minicomputers, mainframe computers, distributed computing environments that include any of the above systems or devices, and the like. Such systems or devices may accept input from the user in any way, including from input devices such as a keyboard or pointing device, via gesture input, proximity input (such as by hovering), and/or via voice input.

Examples of the disclosure may be described in the general context of computer-executable instructions, such as program modules, executed by one or more computers or other devices in software, firmware, hardware, or a combination thereof. The computer-executable instructions may be organized into one or more computer-executable components or modules. Generally, program modules include, but are not limited to, routines, programs, objects, components, and data structures that perform particular tasks or implement particular abstract data types. Aspects of the disclosure may be implemented with any number and organization of such components or modules. For example, aspects of the disclosure are not limited to the specific computer-executable instructions or the specific components or modules illustrated in the figures and described herein. Other examples of the disclosure may include different computer-executable instructions or components having more or less functionality than illustrated and described herein.

In examples involving a general-purpose computer, aspects of the disclosure transform the general-purpose computer into a special-purpose computing device when configured to execute the instructions described herein.

When used in a LAN networking environment, the computer 710 is connected to the LAN 772 through a network interface controller or adapter 774. When used in a WAN networking environment, the computer 710 typically includes a modem 775 or other means for establishing communications over the WAN 773, such as the Internet. The modem 775, which may be internal or external, may be connected to the system bus 730 via the user input interface 760 or other appropriate mechanism. A wireless networking component such as comprising an interface and antenna may be coupled through a suitable device such as an access point or peer computer to a WAN or LAN. In a networked environment, program modules depicted relative to the computer 710, or portions thereof, may be stored in the remote memory storage device. By way of example, and not limitation, FIG. 7 illustrates remote application programs 776 as residing on memory device 771. It may be appreciated that the network connections shown are exemplary and other means of establishing a communication link between the computers may be used.

The examples illustrated and described herein as well as examples not specifically described herein but within the scope of aspects of the disclosure constitute an example appliance management environment. For example, the elements illustrated in FIG. 1, FIG. 4, FIG. 5, FIG. 6 and FIG. 7, such as when encoded to perform the operations illustrated in in FIG. 2, FIG. 3, FIG. 5, FIG. 6 and FIG. 7, constitute an example means for generating a flavor profile pertaining to an item (e.g., sensors).

The order of execution or performance of the operations in examples of the disclosure illustrated and described herein is not essential, unless otherwise specified. That is, the operations may be performed in any order, unless otherwise specified, and examples of the disclosure may include additional or fewer operations than those disclosed herein. For example, it is contemplated that executing or performing a particular operation before, contemporaneously with, or after another operation is within the scope of aspects of the disclosure.

When introducing elements of aspects of the disclosure or the examples thereof, the articles “a,” “an,” “the,” and “said” are intended to mean that there are one or more of the elements. The terms “comprising,” “including,” and “having” are intended to be inclusive and mean that there may be additional elements other than the listed elements. The term “exemplary” is intended to mean “an example of.” The phrase “one or more of the following: A, B, and C” means “at least one of A and/or at least one of B and/or at least one of C.”

Having described aspects of the disclosure in detail, it will be apparent that modifications and variations are possible without departing from the scope of aspects of the disclosure as defined in the appended claims. As various changes could be made in the above constructions, products, and methods without departing from the scope of aspects of the disclosure, it is intended that all matter contained in the above description and shown in the accompanying drawings shall be interpreted as illustrative and not in a limiting sense.

In some embodiments, a computing system for determining a flavor profile of an item comprises a memory device storing computer-executable instructions and a processor configured to execute the computer-executable instructions to obtain a spectral profile associated with the item, identify at least one attribute value for at least one attribute of the item based on the received spectral profile, determine a flavor score for the item based on the at least one attribute value, obtain time series data associated with the item corresponding to a number of days, calculate a predicted flavor score for the item relative to the number of days based on the received time series data, and generate a flavor profile for the item based at least on the predicted flavor score.

In some embodiments, an apparatus and a corresponding method performed by the apparatus, comprises obtaining a spectral profile of the item, identifying at least one attribute value of at least one attribute of the item based on the received spectral profile, obtaining time series data associated with the item corresponding to a number of days, calculating a predicted flavor score for the item relative to the number of days based on the obtained time series data, and generating a flavor profile for the item based on the flavor score and the item data.

In some embodiments, a computer storage medium having computer-executable instructions stored thereon for generating a flavor profile for an item, that upon execution by a processor, causes the processor to obtain a spectral profile associated with the item, determine at least one attribute and a corresponding score of the at least one attribute of the item based on the obtained spectral profile, obtain time series data associated with the item corresponding to a number of days, calculate a predicted attribute score of the at least one attribute based on the corresponding score of the attribute and the received time series data, determine a flavor score of the item based on the calculated predicted attribute score, and generate a flavor profile for the item based at least on the flavor score.

Those skilled in the art will recognize that a wide variety of other modifications, alterations, and combinations can also be made with respect to the above described embodiments without departing from the scope of the invention, and that such modifications, alterations, and combinations are to be viewed as being within the ambit of the inventive concept.

What is claimed is:

1. A system for determining a flavor profile of an item, the system comprising:
a hyperspectral camera, wherein the hyperspectral camera is configured to capture spectral images of an item;
a diffuse lighting system, wherein the diffuse lighting system is configured to provide indirect illumination for the item; and
a computing system, wherein the computing system comprises:
a memory device storing computer-executable instructions; and
a processor configured to execute the computer-executable instructions to:
obtain the spectral images of the item;
generate, based on the spectral images of the item, a spectral profile for the item;
identify at least one attribute value for at least one attribute of the item based on the spectral profile;
determine a flavor score for the item based on the at least one attribute value;
obtain time series data associated with the item corresponding to a number of days;
calculate a predicted flavor score for the item relative to the number of days based on the received time series data; and
generate a flavor profile for the item based at least on the predicted flavor score.

2. The system of claim 1, wherein the at least one attribute comprises at least one of acidity, crunchiness, ripeness, firmness, and sugar levels.

3. The system of claim 1, wherein the processor is further configured to execute the computer-executable instructions to generate the flavor profile for each day of the number of days.

4. The system of claim 1, wherein the processor is further configured to execute the computer-executable instructions to obtain sensor data via at least one input device.

5. The system of claim 1, wherein the processor is further configured to execute the computer-executable instructions to calculate the predicted flavor score based on the identified at least one attribute value and at least a second attribute value of the item.

6. The system of claim 1, wherein the processor is further configured to execute the computer-executable instructions to update the flavor profile at predetermined intervals.

7. The system of claim 1, further comprising:
one or more sensors, wherein the one or more sensors are configured to capture sensor data for the item.

8. The system of claim 1, wherein the processor is further configured to execute the computer-executable instructions to receive transportation data associated with transportation of the item post-packing via at least one input device, and to update the flavor profile based at least in part on the received transportation data.

9. The system of claim 1, wherein the flavor profile further comprises information regarding origination of the item.

10. The system of claim 1, wherein the flavor profile further comprises information regarding transportation of the item.

11. The system of claim 1, wherein the flavor profile further comprises information regarding quantity of the item available.

12. The system of claim 1, wherein the processor is further configured to execute the computer-executable instructions to determine at least one of the following based on the flavor profile: priority of placement of the item in a store from an inventory of an item type associated with the item, selection of a distribution center for the item, pricing of the item, inspection criteria for the item, safety stock number of the item at the store, shelf life of the item, quality metrics associated with different suppliers of the item type, and impact of weather on the quality of the item type.

13. A computer-implemented method for generating a flavor profile for an item, the computer-implemented method comprising:
providing, by a diffuse lighting system, indirect illumination for an item;
obtaining, by a hyperspectral camera, spectral images of the item;
obtaining, from the hyperspectral camera, the spectral images of the item;
generating, based on the spectral images of the item, a spectral profile of the item;
identifying at least one attribute value of at least one attribute of the item based on the spectral profile;
obtaining time series data associated with the item corresponding to a number of days;
calculating a predicted flavor score for the item relative to the number of days based on the obtained time series data;
generating a flavor profile for the item based on the flavor score and the item data.

14. The computer-implemented method of claim 13, wherein the at least one attribute comprises at least one of acidity, crunchiness, and sugar levels.

15. The computer-implemented method of claim 13, further comprising:
generating the flavor profile for each day of the number of days.

16. One or more computer storage media having computer-executable instructions stored thereon for generating a flavor profile for an item, that upon execution by a processor, causes the processor to:
provide, by a diffuse lighting system, indirect illumination for an item;
capture, by a hyperspectral camera, spectral images of the item;
generate, based on the spectral images of the item, a spectral profile for the item;
determine at least one attribute and a corresponding score of the at least one attribute of the item based on the spectral profile;
obtain time series data associated with the item corresponding to a number of days;
calculate a predicted attribute score of the at least one attribute based on the corresponding score of the attribute and the received time series data;
determine a flavor score of the item based on the calculated predicted attribute score;
generate a flavor profile for the item based at least on the flavor score.

17. The one or more computer storage media of claim 16, wherein the flavor profile is accessed via a bar code or QR code on a packing material associated with the item.

18. The one or more computer storage media of claim 16, wherein the flavor profile further comprises information regarding origination of the item.

19. The one or more computer storage media of claim 16, wherein the processor is further configured to execute the computer-executable instructions to determine at least one of the following based on the flavor profile: priority of placement of the item in a store from an inventory of an item type associated with the item, selection of a distribution center for the item, pricing of the item, inspection criteria for the item, safety stock number of the item at the store, shelf life of the item, quality metrics associated with different suppliers of the item type, and impact of weather on the quality of the item type.

20. The one or more computer storage media of claim 16, wherein the processor is further configured to execute the computer-executable instructions to generate the flavor profile for each day of the number of days.
